(19) United States
(12) Patent Application Publication
(10) Pub. No.: US 2025/0368969 A1
(43) Pub. Date: Dec. 4, 2025

(54) LACTATE OXIDASE VARIANTS AND THEIR USES FOR LACTATE DETECTION

(71) Applicant: ASSEMZYME CO., LTD., Taipei City (TW)

(72) Inventors: Yaw-Kuen LI, Hsinchu City (TW); Chin-Wen FU, New Taipei City (TW); Ji-Hua XIE, Taipei City (TW); Chang-Ching WENG, Taipei City (TW); Wan-Ting HUANG, Taipei City (TW)

(21) Appl. No.: 18/875,200

(22) PCT Filed: Jul. 28, 2022
(86) PCT No.: PCT/CN2022/108437

§ 371 Date: Dec. 16, 2024

(51) Int. Cl. C12N 9/02; C12Q 1/00; C12Q 1/26

(52) U.S. Cl. CPC C12N 9/0069; C12Q 1/005; C12Q 1/26; C12Y 113/12004; G01N 2333/90241

(57) ABSTRACT

Disclosed herein is directed to a lactate oxidase variant that exhibits reduced affinity to lactate as compared to a wild-type lactate oxidase. The lactate oxidase variant has one or more amino acid substitutions occurring at positions 95, 96, and/or 175 of the wild-type amino acid sequence. Also disclosed herein is a method for detecting and quantifying lactate in various liquid samples by using the present lactate oxidase variant. The method mainly includes steps of contacting the liquid sample with said lactate oxidase variant; measuring a current generated by the reaction between the lactate oxidase variant and the lactate in the liquid sample; and determining the concentration of lactate in the liquid sample by interpolating or extrapolating the current with that of a control sample having a known concentration of lactate.

### BACKGROUND OF THE INVENTION

### 1. Field of the Invention

The present disclosure relates to lactate oxidase variants and their uses for lactate detection. More particularly, the present disclosure relates to lactate oxidase variants and their uses for electronically detecting and quantifying lactate in a liquid sample.

### 2. Description of Related Art

Lactate is a key metabolite of the anaerobic metabolic pathway in cells, therefore may serve as clues for monitoring multiple physiological processes in various organisms. Thus, lactate concentration has been widely used as a crucial parameter for assessing patient's health condition in clinical diagnostics and for continuous surveillance in food and fermentative industries.

In clinical diagnostics, an increase in lactate concentration resulted from uninterrupted anaerobic metabolism causes accumulation of lactic acid, which inevitably results in lactic acidosis as one of symptoms of severe sepsis. Therefore, blood lactate levels in patients act as alarm signals for the severity of illness, improving the diagnosis and treatments of a broad range of diseases. Lactate also is a significant factor in sports medicine, especially for determining physical fitness in athletics. Since elevated levels of blood lactate result in decrease of pH level in blood and eventually resulting in fatigue. The blood level of lactate during exercise is used as an indicator for the evaluation of athletic training status and fitness.

Lactate estimation also can be found in food and fermentative industries. Lactate is produced during fermentation of foods, therefore is used for detecting the presence of microorganism fermentation in fermented food products such as fermented milk products, wine, cured meat and fish, and pickled vegetables, thus, it serves as an indicator for the freshness and quality of the food.

Various methods for determining lactate levels have been developed; among them, high performance liquid chromatography (HPLC) is the most common approach. Other analytical methodologies including fluorometry, colorimetric test, chemiluminescence and magnetic resonance spectroscopy are commonly employed as well. However, these approaches suffer from drawbacks like time-consuming processes and costly machinery and trained manpower.

Portable and disposable biosensors are currently developed to overcome these limitations. Typically, biosensing methods possess the advantages of being simple and direct, combining rapid response with high specificity, economical and are user friendly. Ideally, the lactate concentration in biological fluids such as whole blood, sweat, and saliva can be determined by biosensors; however, biosensors currently on the market are only adapted for blood lactate detection, which requires an invasive method of sample collection, thus, is not popular among users nor can be used for detecting lactate in samples other than blood.

Take human sweat as an example, the range of lactate concentration is wider and varied more than those in blood (in some cases, it increases up to 80 mM after exercise, whereas the lactate level in blood rises up to 25 mM during exertion), resulting a poor accuracy for conventional lactate meters. Further, all current biosensors need a large fluid volume for their operation, and the differences in pH values between blood and sweat also bring difficulties for blood lactate meter to be used to detect lactate concentration in sweat.

In view of the foregoing, there exists in the related art a need of an improved tool and approach for continuously detecting the lactate level in human body fluids in a non-invasive and efficient way.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a lactate oxidase variant that exhibits reduced affinity to lactate, yet gives rise to a wider detectable range, derived from a wild-type lactate oxidase of SEQ ID NO:1, wherein the lactate oxidase variant comprises an amino acid substitution at positions 95, 96, or 175 of the SEQ ID NO: 1, or a combination thereof. In the present lactate oxidase variant, alanine (A) at position 95 of the SEQ ID NO: 1 is substituted by asparagine (N) or glutamine (Q); alanine (A) at position 96 of the SEQ ID NO: 1 is substituted by cysteine (C); and/or seine(S) at position 175 of the SEQ ID NO: 1 is substituted by cysteine (C).

According to one embodiment of the present disclosure, the lactate oxidase variant comprises the amino acid sequence of the SEQ ID NO: 1 in which the alanine (A) at position 95 thereof is substituted by asparagine (N).

According to an alternative embodiment of the present disclosure, the lactate oxidase variant comprises the amino acid sequence of the SEQ ID NO:1 in which the alanine (A) at position 95 thereof is substituted by glutamine (Q).

According to another embodiment of the present disclosure, the alanine (A) at position 96 of the SEQ ID NO: 1 is substituted by cysteine (C)

According to still another embodiment of the present disclosure, the seine(S) at position 175 of the SEQ ID NO:1 is substituted by cysteine (C).

Alternatively or optionally, the cysteine (C) at position 175 of the SEQ ID NO:1 may be carboxymethylated.

According to preferred embodiments of the present disclosure, the lactate oxidase variant has the amino acid sequence of SEQ ID NO: 2, 3, 4, 5, or 6.

In some embodiments of the present disclosure, the present lactate oxidase variant has a binding affinity to lactate lower than that of the wild-type lactate oxidase.

Another aspect of the present disclosure is directed to a method for detecting and quantifying lactate in a liquid sample. The method comprises steps of (a) contacting the liquid sample with the aforementioned lactate oxidase variant; (b) measuring a current generated by the reaction between the aforementioned lactate oxidase variant and the lactate in the liquid sample; and (c) determining the concentration of lactate in the liquid sample via interpolating or extrapolating the current measured in step (b) with that of a control sample having a known concentration of lactate.

According to some embodiments of the present disclosure, the liquid sample has pH value ranging from 4 to 9.

According to some embodiments of the present disclosure, the liquid sample has a salinity between 0 to 1000 mM.

In some preferred embodiments, the liquid sample is sweat.

According to some embodiments of the present disclosure, the method is capable of detecting lactate ranging from 0 to 300 mM in the liquid sample.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A is a line graph depicting the calibration curve of lactate concentrations vs. currents established in accordance with the present method conducted in phosphate buffer (PB) solution (pH 5.0) via use of the present mutant lactate oxidase A95Q;

FIG. 1B is a line graph depicting the calibration curve of lactate concentrations vs. currents established in accordance with the present method conducted in PB solution (pH 7) via use of the present mutant lactate oxidase A95Q;

FIG. 2A is a line graph depicting the calibration curve of lactate concentrations vs. currents established in accordance with the present method conducted in PB solution (pH 5.0) via use of the present mutant lactate oxidase A96C;

FIG. 2B is a line graph depicting the calibration curve of lactate concentrations vs. currents established in accordance with the present method conducted in PB solution (pH 7.0) via use of the present mutant lactate oxidase A96C;

FIG. 2C is a line graph depicting the calibration curve of lactate concentrations vs. currents established in accordance with the present method conducted in synthetic sweat (pH 5.0) via use of the present mutant lactate oxidase A96C;

FIG. 3A is a plot depicting the relationship between lactate concentrations and currents

measured in accordance with the present method conducted in PB solution (pH 5.0) via use of the present mutant lactate oxidase S175C;

FIG. 3B is a plot depicting the relationship between lactate concentrations and currents measured in accordance with the present method conducted in PB solution (pH 7.0) via use of the present mutant lactate oxidase S175C;

FIG. 4A is a plot depicting the relationship between lactate concentrations and currents measured in accordance with the present method conducted in PB solution (pH 5.0) via use of the present carboxymethylated S175C;

FIG. 4B is a plot depicting the relationship between lactate concentrations and currents measured in accordance with the present method conducted in PB solution (pH 7.0) via use of the present carboxymethylated S175C;

FIG. 5 is a line graph depicting calibration curves of lactate concentrations vs. currents established in accordance with the present method conducted in synthetic sweat (pH 5.0) via use of different amount of the present A96C;

FIG. 6 is a line graph depicting calibration curves of lactate concentrations vs. currents established in accordance with the present method conducted in synthetic sweat having various PH levels (pH 5.0-7.0) via use of the present A96C; and

FIG. 7 is a plot depicting the detected currents corresponding to a certain lactate concentration (40 mM) in synthetic sweat samples having various salinities (0 to 800 mM) and pH levels via use of the present A96C.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

Typically, a term of “wide-type” is used to describe a gene or a protein when it is found in its natural, non-mutated (unchanged) form. The term “wild-type lactate oxidase” as used herein refers a form of lactate oxidase protein typically occurs in nature (i.e., bacteria) without genetic, structural, and/or functional change. Specifically, the wild-type form of lactate oxidase is a full-length native lactate oxidase having 374 amino acids set forth as SEQ ID NO: 1.

The term “lactate oxidase variant(s)” as used herein is intended to encompass one or more forms of the lactate oxidase polypeptide derived from wild-type lactate oxidase by substitution, in which at least one amino acid in the wild-type lactate oxidase sequence was replaced by another amino acid. The term of “lactate oxidase variant” alternatively or optionally refers to a form of lactate oxidase peptide in which one or more residues have been subjected to post-translational modification (PTM) and/or chemical modification to increase functional diversity of the proteome. Types of PTMs include phosphorylation, methylation, acetylation, ubiquitination, hydroxylation, succinylation, glycosylation, and SUMOylation, but not limited thereto; and exemplary chemical modification includes but is not limited to carboxymethylation. In the present disclosure, the modification made to amino acid residues is carboxymethylation. Well-known and commonly used designations may be interchangeably used herein to indicate the same mutation occurring on peptide sequences. According to the present disclosure, for example, a substitution from alanine (A) at position 95 to asparagine (N) can be indicated as 95A, A95, A95N, or Ala95Asp.

The term “binding affinity” used herein refers to the strength of the sum total of non-covalent interactions between a single binding site of a substrate (i.e., lactate and/or lactic acid) and an enzyme (e.g., lactate oxidase or its mutated variants). The affinity of an enzyme for a substrate can generally be thought to be related to Michaelis Constant (Kₘ), which describes the substrate concentration at which half the enzyme's active sites are occupied by the substrate. If Kₘ is less, stronger binding affinity for the substrate. Generally, compared with the wild-type enzyme, the binding affinity of its mutated form may or may not be changed, depending on where the mutation occurs. According to the present disclosure, the binding affinities of the present lactate oxidase variants for lactate and/or lactic acid decline, compared to that of the wide-type lactate oxidase.

The term “liquid sample” used herein refers to a sample collected and/or obtained from natural environments or artificial products as a liquid form that may or may not contain lactate and/or lactic acid, and the solvent is mostly water. The liquid sample used in the present disclosure can be a bio-sample having metabolic products (i.e., lactate and/or lactic acid) of organisms. Examples of bio-sample suitable for use in the present disclosure include body fluids of a mammal, more preferably a human (e.g., sweat, urine, saliva, blood, and interstitial fluids); and fermented liquids produced by microorganisms (e.g., fermented foods and rancid foods). The liquid samples can contain one or more substances including but not limiting to minerals, trace elements, metal ions and/or heavy metal ions, metabolite, excretion, microplastics, micronekton, and microorganisms. In addition, the liquid sample has a variety of measurable parameters including but not limited to pH value and salinity.

### 2. Detail Description of Preferred Embodiments

The present disclosure is based, at least in part, on the discovery of some lactate oxidase variants possess a binding affinity to lactate/lactic acid lower than that of a wild-type lactate oxidase, thus are capable of detecting concentrated lactate in high sensitivity without being interfered by pH or salinity. Further, a current is generated upon reaction of the enzyme (i.e., lactate oxidase) and the substrate (i.e., lactate, lactic acid, or a combination thereof) in the presence of an electric field, and it was unexpectedly found that a linear relationship exists between high concentrations of lactate (e.g., >20 mM) and the current, thus said current may serve as an indicator for lactate detection.

### 2.1 Lactate Oxidase Variants

The first aspect of the present disclosure pertains to a lactate oxidase variant, which

comprises at least one amino acid mutation that leads to a reduced binding affinity to lactate/lactic acid as compared to that of a wild-type lactate oxidase. The lactate oxidase variant has an amino acid sequence derived from the wild-type lactate oxidase set forth as SEQ ID NO:1, in which one or more amino acid(s) is/are substituted at positions 95, 96, and/or 175 of SEQ ID NO:1. Specifically, alanine (A) at position 95 of the SEQ ID NO:1 is substituted by asparagine (N) or glutamine (Q); alanine (A) at position 96 of the SEQ ID NO:1 is substituted by cysteine (C); and/or seine(S) at position 175 of the SEQ ID NO:1 is substituted by cysteine (C).

In accordance with the embodiments of the present disclosure, the present lactate oxidase may have an amino acid sequence at least 99% identical to SEQ ID NO: 1, such as having 99.1%, 99.2%, 99.3%, 99.4%, 99.5%, 99.6%, 99. 7%, 99.8%, and 99.9% sequence identity to SEQ ID NO: 1; preferably, an amino acid sequence at least 99.2% identical to SEQ ID NO: 1; more preferably, an amino acid sequence at least 99.8% identical to SEQ ID NO: 1, with at least one amino acid substitution occurs at positions 95, 96, or 175 of the SEQ ID NO: 1, and such amino acid substitution is selected from the group consisting of A95N, A95Q, A96C, S175C and a combination thereof.

According to some embodiments of the present disclosure, the present lactate oxidase variant termed as A95N may have an amino acid sequence at least 99.8% identical to SEQ ID NO: 1, in which alanine (A) at position 95 of the SEQ ID NO: 1 is substituted by asparagine (N). Accordingly, the lactate oxidase A95N variant has the amino acid sequence of SEQ ID NO: 2.

According to other embodiments of the present disclosure, the present lactate oxidase variant termed as A95Q may have an amino acid sequence at least 99.8% identical to SEQ ID NO: 1, in which alanine (A) at position 95 of the SEQ ID NO: 1 is substituted by glutamine (Q). Accordingly, the lactate oxidase A95Q variant has the amino acid sequence of SEQ ID NO: 3.

According to other embodiments of the present disclosure, the present lactate oxidase variant termed as A96C may have an amino acid sequence at least 99.8% identical to SEQ ID NO: 1, in which alanine (A) at position 96 of the SEQ ID NO: 1 is substituted by cysteine (C). Accordingly, the lactate oxidase A96C variant has the amino acid sequence of SEQ ID NO: 4.

According to still other embodiments of the present disclosure, the present lactate oxidase variant termed as S175C may have an amino acid sequence at least 99.8% identical to SEQ ID NO: 1, in which seine(S) at position 175 of the SEQ ID NO: 1 is substituted by cysteine (C). Accordingly, the lactate oxidase S175C variant has the amino acid sequence of SEQ ID NO: 5.

The lactate oxidase variants of the present disclosure may be prepared by substitution or modification using genetic or chemical methods well known in the art. Genetic methods may include site-specific mutagenesis of the encoding DNA sequence, polymerase chain reaction (PCR), gene synthesis, CRISPR/cas9 gene editing, and the like. The correct nucleotide changes may be verified for example by sequencing. The nucleotide sequence of native bacterial (e.g., Aerococcus viridans) lactate oxidase is available from public database such as UniProtKB (Aerococcus viridans ATCC 11563; accession code: D4YFm2). The amino acid sequence of wild-type lactate oxidase is shown in SEQ ID NO: 1.

The present lactate oxidase variants may be produced, for example, by solid-state peptide synthesis or recombinant production. For recombinant production, one or more polynucleotides encoding said lactate oxidase variants are independently isolated and inserted into suitable vector(s) for further cloning and/or expression in a host cell, mostly is Escherichia coli. Such polynucleotide may be readily isolated and sequenced using conventional procedures. Methods which are well known to those skilled in the art may be used to construct expression vectors containing the coding sequence of the present lactate oxidase variants along with appropriate transcriptional/translational control signals. Examples of these methods include, but are not limited to, in vitro recombinant DNA techniques, synthetic techniques, and in vivo recombination/genetic recombination. The expression vector may be part of a plasmid, virus, or may be a nucleic acid fragment. Typically, the expression vector is an expression cassette into which the polynucleotide encoding the present lactate oxidase variant is cloned in operable association with a promoter and/or other transcription or translation control elements, which may be operably associated with a nucleic acid encoding a polypeptide, if the promoter is capable of effecting transcription of that nucleic acid. According to some embodiments of the present disclosure, site-directed mutagenesis on native lactate oxidase is expressed and performed via use of the pRSET expression vector and conventional tools well known in the art.

Alternatively or optionally, the present lactate oxidase variant may be further subjected to post-translation modification (PTM) and/or chemical modification, in which additional functional groups are introduced thereon the residues. Exemplary PTMs include, but are not limited to, phosphorylation, glycosylation, ubiquitination, s-nitrosylation, methylation, acetylation, hydroxylation, succinylation, and SUMOylation. Exemplary chemical modification includes but is not limited to carboxymethylation. PTMs and/or chemical modification for a protein can be performed by any methods and tools well known in the art depending on practical needs and desired purposes. According to one embodiment of the present disclosure, the present lactate oxidase variant is subjected to carboxymethylation via reacting with iodoacetate (IA) or iodoacetic acid (IAA), which binds covalently with the thiol group of cysteine (C), thereby creates a carboxyl-methyl group thereon. In one working example, the present lactate oxidase variant S175C is subjected to chemical modification by reacting with iodoacetic acid thereby producing a carboxymethylated cysteine residue. Accordingly, the carboxymethylated lactate oxidase S175C has the amino acid sequence of SEQ ID NO: 6.

In certain embodiments, the amino acid substitution and/or modification made to wide-type lactate oxidase may results in a decrease in the binding affinity of enzyme (i.e., lactate oxidase) to substrate (i.e., lactate/lactic acid) by at least 50%, such as by at least 30%, 25%, 20%, 10%, 7%, 5%, 2%, or even 1%. The binding affinity of the present lactate oxidase variant to its substrate can be measured or determined by various assays known in the art, such as colorimetric assay, in which the kinetic parameters (e.g., Kₘ and Vₘₐₓ in Michaelis-Menten equation) for each lactate oxidase variant is determined by following the well-established procedures known in the art.

2.2 Methods for detecting and Quantifying Lactate

Another aspect of the present disclosure is directed to a method for detecting and quantifying lactate in a liquid sample. The method comprises at least following steps:
(a) contacting the liquid sample with the present lactate oxidase variant set forth in previous section;
(b) measuring a current generated by the reaction between the present lactate oxidase variant and the lactate in the liquid sample; and
(c) determining the concentration of lactate in the liquid sample via interpolating or extrapolating the current measured in step (b) with that of a control sample having a known concentration of lactate.

According to the present disclosure, the current generated by the reaction between the present lactate oxidase variant of step (a) and the lactate in the liquid sample may be measured with the aid of an electrode system. In this regard, before commencing the present method, it is preferable to construct a standard calibration curve for lactate detection by measuring the currents generated between the lactate oxidase and various known concentrations of lactate. The electrode system typically includes, in its structure, a working electrode and a counter electrode, and optionally a reference electrode. Exemplary materials suitable for constructing working and/or counter electrodes include, but are not limited to, carbon (e.g., pyrolytic carbon, graphite, graphene, glassy carbon, carbon paste, perfluorocarbon (PFC), or the like) and metals (e.g., platinum, gold, silver, nickel, palladium, or the like). Additionally, exemplary reference electrode may be saturated calomel electrode, or silver/silver chloride electrode. The electrode system can be made of any designated materials as exemplified above by methods well known in the art, for instance, photolithography vapor deposition, sputtering, or printing (e.g., screen printing, gravure printing, flexographic printing, and the like). In one working example, the electrode system of the present disclosure is a screen-printed carbon electrode (SPCE) made of graphite and graphene.

For the purpose of lactate detection, the present lactate oxidase variants are deposited onto the surface of the electrodes system described above (e.g., SPCE) in the presence of a redox mediator. According to working embodiments, the present lactate oxidase variants and the redox mediator are mixed at a designated ratio to form a mixture, which is then immobilized on the surface of SPCE by electrodeposition or drop-casting, thereby producing a lactate sensor suitable for use in the present method.

Example of redox mediator suitable for use in the present method includes, but is not limited to, poly(aniline)-poly(acrylate), poly(aniline)-poly(vinyl sulfonate), poly(pyrrole), poly(pyrrole) poly(vinyl sulfonate), poly(vinylpyrrolidone), poly(1-vinylimidazole) (PVIm), ferricyanide salts, ferrocyanide salts, cobalt phthalocyanine, hydroxymethyl ferrocene, osmium (Os) complexes, [7-(dimethylamino)-4-nitrophenothiazin-3-ylidene]-dimethylazanium chloride, benzo [a] phenoxazin-9-ylidene(dimethyl)azanium, tetrathiafulvalene, and a copolymer or a combination thereof. In one working example, the redox mediator is a copolymer of poly (1-vinylimidazole) and an osmium complex (PVImQOs); in another working example, the redox mediator is potassium ferricyanide (K₃[Fe(CN)₆]).

For the purpose of establishing a calibration curve, a control sample having a known

concentration of lactate or lactic acid is contacted with the lactate sensor with a fixed electric potential being applied thereon, such that a current generated by an electrochemical reaction between the lactate and the present lactate oxidase variants immobilized on the electrodes can be detected by any means known in the art, specifically an electrochemical analyzer. Accordingly, a standard calibration curve can be established based on the detected currents corresponding to the known concentrations of lactate. In some embodiments of the present disclosure, the known concentration of lactate ranges from about 0 to 300 mM; for example, 0, 0.1, 0.2, 0.3, 0.4, 0.5, 0.6, 0.7, 0.8, 0.9, 1, 1.5, 2, 2.5, 3, 4, 5, 6, 7, 8, 9, 9.9, 10, 14.8, 19.6, 20, 29.1, 30, 38.5, 40, 47.6, 50, 56.6, 60, 65.4, 70, 74.1, 80, 82.6, 90, 90.9, 100, 110, 120, 130, 130.4, 140, 150, 160, 166.7, 170, 180, 190, 200, 210, 220, 230, 240, 250, 259.3, 260, 270, 280, 290, or 300 mM. In one working example, the calibration curve is constructed with lactate at the concentrations of 0.2, 0.5, 1, 2, 5, 10, 15, 20, 30, 38.5, 47.6, 56.6, 65.4, 74.1, 82.6, 90.9, 130.4, and 166.7 mM. In another working example, the calibration curve is constructed with lactate at the concentrations of 0.2, 0.5, 1, 2, 5, 9.9, 19.6, 29.1, 47.6, 90.9, 130.4, 166.7, 200, and 259.3 mM. In still another working example, the calibration curve is constructed with lactate at the concentrations of 0.2, 0.4, 0.6, 0.8, 1, 1.5, 2, 2.5, 3, 4, 5, 6, 7, 8, and 10 mM. In still another working example, the calibration curve is constructed with lactate at the concentrations of 0, 5, 10, 20, 30, 40, 50, 60, 70, 80, 90, 100, 120, and 200 mM. According to embodiments of the present disclosure, the control sample is a mimic of human sweat; preferably, the control sample used in the present method is synthetic sweat prepared in accordance with international standards. Alternatively or optionally, the control sample used in the present method specifically mimics the pH, osmolarity, and ion concentrations of human fluids; preferably, the control sample is a phosphate buffer.

In step (a) of the present method, a liquid sample (e.g., human sweat, a buffer and etc) is contacted with the present lactate oxidase variants immobilized on the surface of the electrode system for at least 5, 10, 20, or 60 seconds, as long as it is sufficient enough to generate an electrochemical current resulted from a reaction between the enzymes and the substrates. Then, in step (b), said current is measured and determined by any means known in the art, such as electrochemical analyzer set forth above.

According to some embodiments of the present disclosure, the liquid sample has a pH value between 4 to 9; for example, a pH value of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9. In one working example, the liquid sample has the pH value of 5.0, 6.0, or 7.0. According to alternative embodiments of the present disclosure, the liquid sample has a salinity of 0 to 1000 mM; for example, a salinity of 0, 100, 200, 300, 400, 500, 600, 700, 800, 900, or 1000 mM. In another working example, the liquid sample has a salinity of 0, 100, 200, 300, 400, 500, 600, 700, or 800 mM. The liquid sample suitable for use in the present method may be derived from natural environments (e.g., animal bodies) or artificial products (e.g., food products).). Examples of the liquid sample suitable for use in the present method include, but are not limited to, sweat, urine, saliva, blood, interstitial fluids, and fermented liquids produced by microorganisms. In one working example, the liquid sample is sweat.

In the final step of the present method, i.e., step (c), the concentration of lactate in liquid sample can be determined from the calibration curve by interpolation or extrapolation. Specifically, the measured current in step (b) is substituted into the calibration curve constructed based on known concentrations of lactate in the control sample prior to step (a) as described above, thereby obtaining the concentration of lactate in the liquid sample.

Taken together, the present method comprises at least, the steps (a) to (c) as described above, in which the present method is capable of detecting lactate in any liquid sample having a trace or an abundant quantity of lactate. According to the present disclosure, the present method is capable of detecting lactate ranging from 0 to 300 mM; for example, ranging from 0 to 280 mM, from 0 to 260 mM, from 0 to 250 mM, from 0 to 200 mM, from 0 to 150 mM, from 0 to 130 mM, from 0 to 120 mM, from 0 to 110 mM, from 0 to 100 mM, from 0.2 to 5 mM, from 0.2 to 10 mM, from 0.2 to 166 mM, from 0.2 to 180 mM, from 0.2 to 260 mM, from 0.5 to 90 mM, from 0.5 to 100 mM, from 0.5 to 110 mM, from 0.5 to 120 mM, or from 5 to 100 mM. In some preferred examples, the present method is capable of detecting lactate over 100 mM in liquid sample.

By the virtue of the above features, the present method can detect and quantify lactate concentration, particularly the abundant lactate in aqueous environments, which was unable to be detected by conventional detecting methodologies. In addition, the present method is capable of detecting lactate in aqueous samples that also contain a variety of substances, therefore can be applied in diverse liquid sources.

### EXAMPLES

### Materials and Methods

### Gene Synthesis and Mutagenesis

Escherichia coli BL21 (DE3) were transformed with expression vectors respectively expressing wild-type and mutant lactate oxidases. The lactate oxidase gene was cloned based on the wild-type sequence of Aerococcus viridans ATCC 11563 retrieved from database UniProtKB (Accession code: D4YFm2), in which the wild-type sequence was inserted into multi-cloning site of the expression vector pRSET in accordance with the preferential use of codon in E. coli. Site-directed mutagenesis was performed using conventional protocols provided along with a commercial mutagenesis kit (Quikchange, Agilent, US) with primer sets listed in Table 1. The plasmid sequences were confirmed by 3730XL DNA Analyzer (Thermo Fisher Scientific, USA).

TABLE 1
Primer sequences

| Name | | SEQ |
|---|---|---|
| of the | Nucleotide sequences | ID |
| Primer | (5′→3′) | NO |
| A95N-F | CCGTTTATTATGGCACCGATTA | 7 |
| | ACGCACATGGTCTGGCACATAC | |
| A95N-R | GTATGTGCCAGACCATGTGCGT | 8 |
| | TAATCGGTGCCATAATAAACGG | |
| A95Q-F | CGTTTATTATGGCACCGATTCA | 9 |
| | AGCACATGGTCTGGCACAT | |
| A95Q-R | ATGTGCCAGACCATGTGCTTGA | 10 |
| | ATCGGTGCCATAATAAACG | |
| A96C-F | TTATGGCACCGATTGCATGCCA | 11 |
| | TGGTCTGGCACATACCAC | |
| A96C-R | GTGGTATGTGCCAGACCATGGC | 12 |
| | ATGCAATCGGTGCCATAA | |
| S175C-F | TATTCTGACCGCAGATTGCACC | 13 |
| | GTTAGCGGTAATC | |
| S175C-R | GATTACCGCTAACGGTGCAATC | 14 |
| | TGCGGTCAGAATA | |

### Preparation of Wild-Type and Present Mutant Lactate Oxidases

E. coli BL21 (DE3) were transformed by plasmids respectively containing wild-type and lactate oxidases varieties, the transformed DE3 were then inoculated into LB broth containing ampicillin, and cultivated in a shaking incubator (250 rpm) at 37° C. for 16 to 20 hours. 1% of the bacterial culture were taken out and inoculated into the main culture medium: 500 mL of ZYP-5052 medium (0.5% glycerol, 0.05% glucose, 0.2% lactose, 50 mM KH₂PO₄, 25 mM (NH₄)₂SO₄, 50 mM Na₂HPO₄, and 1 mM MgSO₄) containing 100 μg/mL ampicillin. The medium was then cultivated for 8 hours at 37°° C. while shaking. Supernatants were removed by centrifugation (4° C., 5000 g for 20 minutes), and cell pellets were collected and stored at −20° C.

### Purification of Wild-Type and Present Mutant Lactate Oxidases

E coli cells that have been transformed with wild-type or mutant lactate oxidases were resuspended in 50 mL of 50 mM potassium phosphate buffer (PPB, pH 7.0) containing 500 mM NaCl and 20 mM imidazole, and subjected to a homogenizer (NanoLyzer). The obtained cell liquid was centrifuged at 4° C., 10,000 g for 1 hour, and the supernatant was collected. The collected supernatant, which contained recombinant His-tagged proteins as crude cell extracts, were subjected to purification via nickel affinity column and protein purification kit (ÄKTA start, Cytiva). The column was eluted by 5-fold volume of buffer A (pH 7.0, 20 mM imidazole, 500 mM NaCl and 50 mM PPB), the crude cell extracts were loaded into the column, then washed by buffer A again (3-fold volume), and eluted by 20-fold volume of 4 to 100% buffer B (pH7.0, 500 mM imidazole, 500 mM NaCl, and 50 mM PPB), and a total of 20 fractions were collected. After SDS-PAGE analysis, fractions containing target proteins were dialyzed and concentrated by using dialysis membranes (10 kDa). After being replaced with 50 mM Tri-HCl solution (pH 7.0), the purified enzyme solutions were freeze-dried and stored at −20 C.

### Carboxymethylation of Mutant Lactate Oxidases

Solution of purified lactate oxidase variants (80 μL or 3.5 U) was mixed with 0.1 M of iodoacetic acid (IAA) (20 μL) at 37° C. for 60 minutes.

### Enzyme Assays

### Determination of Protein Concentration

Analytes (20 μL) were mixed with 200 μL protein dye (Bradford reagent) for 5 minutes to form a mixture, and O.D. value of the mixture was measured at 595 nm. Concentrations of the proteins were determined based on a calibration curve constructed in accordance with the standard concentrations from 0.05 to 0.3 mg/mL of bovine serum albumin (BSA) solution and their corresponding O.D. values.

### Determination of Protein Activity

### 1. Lactate Oxidative Activity

Diluted enzyme was added into microplates by 50 μL per each well, 50 μL of staining reagent prepared by mixing 0.25 mg/mL of 3, 3′, 5, 5′-tetramethylbenzidine (TMB) and 5 U/mL horseradish peroxidase (HRP), and 50 μL of 10 mM lactic acid were added subsequently into each well, allowing the mixture to react via shaking intermittently at 30° C. for five minutes. The reaction was then blocked by adding 50 μL of 1 M HCl, the absorbance (O.D.) of the solution at the wavelength of 450 nm was determined via use of the molar attenuation coefficient (8) set to 59 cm^{-1.}mmol-1. Lactate oxidative activities (i.e., 1U) were measured at 30° C. at pH 7.0 by the following equation:

$$Activity ( U / mL ) = [ Δ O . D . ( {OD}_{test} - {OD}_{blank} ) \times {V}_{t} \times df ] / ( ε \times t \times l \times Vs ) ,$$

In which Vₜ is the total volume, df is dilution factor, ε is molar attenuation coefficient, t is reaction time, l is light path length (cm), and Vs is enzyme volume.

### 2. Dehydrogenase Activity

Phenazine methosulfate (PMS, 0.5 mM) was mixed with 2,6-dichlorophenol-indophenol (DCIP, 1.2 mM) in potassium phosphate buffer (PPB, 20 mM, pH 7.0) to produce reaction reagent. Diluted enzyme (50 μL in 50 mM PPB, pH 7.0) was added into microplates with 50 μL per well, said reaction reagent (50 μL) and 10 mM lactic acid (50 μL) were added sequentially into each well, the microplate was then subjected to intermittent shaking at 30° C. for five minutes. The absorbance (O.D.) at the wavelength of 600 nm for each well was measured and determined with the molar attenuation coefficient (ε) set to 16.3 cm⁻¹·mmol⁻¹. Lactate dehydrogenase activities (i.e., 1 U) were determined at 30° C., pH 7.0 by the following equation:

$$Activity ( U / mL ) = [ Δ O . D . ( {OD}_{test} - {OD}_{blank} ) \times {V}_{t} \times df ] / ( ε \times t \times l \times Vs ) ,$$

in which Vₜ is total volume, df is dilution factor, ε is molar attenuation coefficient, t is reaction time, l is light path length (cm), and Vs is enzyme volume.

3. Binding affinity

The enzyme kinetic parameters Kₘ, K_{cat}, and Vₘₐₓ of the wild-type or mutant lactate oxidases were determined by reacting with lactate at various concentrations (0.1 to 200 mM for oxidase; 0.1 to 900 mM for dehydrogenase) with each of the present lactate oxidase variants and wild-type lactate oxidase at pH 5.0 or pH 7.0, respectively, and their reaction rates were individually determined based on a slope of a linear plot of time (i.e., reaction interval) against absorbances (O.D.) at wavelength of 450 or 600 nm detected every three seconds within the reaction interval. Kₘ, K_{cat}, and Vₘₐₓ values were obtained afterwards via a calibration curve drawn between the concentrations of lactic acid (taken on x-axis) and the reaction rate (taken on y-axis) under Michaelis-Menten equation.

Preparation of Lactate Sensors with Modified Electrodes

The cyclic voltammetry (CV) analysis was performed with a screen-printed carbon electrode (SPCE, TE100, Zensor) as a three-electrode system, in which the present enzymes were immobilized on the surface of the working electrode via electrodeposition or drop-casting method as described below.

### Electrodeposition

The outersurface of SPCE was washed using ddH₂O and then covered with a mixture of 20 μL of PVImQOs (10 mg/mL) and 100 μL of enzyme solution (4U), which was either the wild-type lactate oxidase or the present lactate oxidase variants (i.e., A95N, A95Q, A96C, S175C, or modified S175C). The SPCE was then subjected to a cyclic voltammetry at 37° C. with a preset electric potential between −1.0V to 0.0V and a preset scan rate of 200 mV/s for 50 cycles, so as to achieve surface modification. The modified SPCE was then placed in PPB solution (pH 5.0, 100 mM) for another cyclic voltammetry with second preset electric potential between −300 mV to 600 mV and scan rate of 60 mV/s for 20 cycles, so as to remove residual PVImQOs. After drying at room temperature, 4 μL of protective agent containing 1% of chitosan and 0.075% of genipin was further added onto the modified SPCE.

### Drop-Casting Method

i. A mixed solution (1 μL) of enzyme solutions (1 to 8 U/μL), potassium ferricyanide (K₃[Fe(CN)₆], 25 to 50 mM), and 0.1% of Triton X-100 was dropped to cover the surface of electrode and dried at room temperature.

ii. Alternatively, 5 μL of PVImQOs (10 mg/mL) and 2.5 μL of enzyme solution were respectively and sequentially dropped and deposited onto the electrode area of SPCE. After drying at 4° C., 4 μL of protective agent containing 1% of chitosan and 0.075% of genipin was further added onto the surface of SPCE. The modified SPCE was dried and stored at room temperature.

### Electrochemical Evaluation

The SPCE was connected to the electrochemical analyzer (ACIP100) combined with the CS100 electrode stand (both Zensor) with a ECP100 (Zensor) cable connector. The voltammograms and the results were recorded and analyzed by the on-device software of ACIP100 (Zensor).

### Lactate Detection

### Sample Preparation

Synthetic sweat was prepared according to the recipe under international standard ISO 3160-2 and the pH value of the synthetic sweat was set to be 5, 6 or 7 by adjusting the amount of sodium hydroxide therein. Potassium phosphate buffer (0.1 M) and its pH value was prepared by adjusting the amount of monobasic and dibasic potassium phosphate that obtained from the supplier (Merck).

### Establishing Calibration Curve

Lactic acid solution at various concentrations (i.e., 0, 0.2, 0.4, 0.5, 0.6, 0.8, 1, 1.5 2, 2.5, 3, 4, 5, 6, 7, 8, 9.9, 10, 14.8, 19.6, 20. 29.1, 30, 38.5, 40, 47.6, 50, 56.6, 60, 65.4, 70, 74.1, 80, 82.6, 90, 90.9, 100, 120, 130.4, 166.7, 200, or 259.3 mM lactic acid in synthetic sweat or phosphate buffer) were respectively applied to the lactate sensor having enzymes immobilized thereon, electric currents generated between electrodes under fixed electric potentials of 300 or 400 mV were measured. Current values at 60th second in reaction generated between blank sample (i.e., 0 mM of lactic acid) and enzymes (i.e., the present mutated lactate oxidases) were recorded as the background, then the buffer solution spiked with said concentrations of lactic acids were respectively applied to the lactate sensor. Each reaction continued for about 20 to 60 seconds and the current of each reaction was recorded at 5 or 10 second, thereby established a calibration curve of current vs. lactate concentration.

### Example 1 Production of the Present Lactate Oxidase Variants

The present five lactate oxidase variants were produced by methods described in the “Materials and Methods” section. The thus produced lactate oxidase variants and their amino acid sequences are listed in Table 2, in which the substitution and modification of designated amino acids are indicated in bold letters.

TABLE 2
The present lactate oxidase variants

| Lactate | | SEQ |
|---|---|---|
| oxidase | | ID |
| variants | Amino acid sequence | NO |
| A95N | MNNNDIEYNAPSEIKYIDVVN | 2 |
| | TYDLEEEASKVVPHGGFNYIA | |
| | GASGDEWTKRANDRAWKHKLL | |
| | YPRLAQDVEAPDTSTEILGHK | |
| | IKAPFIMAPINAHGLAHTTKE | |
| | AGTARAVSEFGTIMSISAYSG | |
| | ATFEEISEGLNGGPRWFQIYM | |
| | AKDDQQNRDILDEAKSDGATA | |
| | IILTADSTVSGNRDRDVKNKF | |
| | VYPFGMPIVQRYLRGTAEGMS | |
| | LNNIYGASKQKISPRDIEEIA | |
| | AHSGLPVFVKGIQHPEDADMA | |
| | IKAGASGIWVSNHGARQLYEA | |
| | PGSFDTLPAIAERVNKRVPIV | |
| | FDSGVRRGEHVAKALASGADV | |
| | VALGRPVLFGLALGGWQGAYS | |
| | VLDYFQKDLTRVMQLTGSQNV | |
| | EDLKGLDLFDNPYGYEY | |
| A95Q | MNNNDIEYNAPSEIKYIDVVN | 3 |
| | TYDLEEEASKVVPHGGFNYIA | |
| | GASGDEWTKRANDRAWKHKLL | |
| | YPRLAQDVEAPDTSTEILGHK | |
| | IKAPFIMAPIQAHGLAHTTKE | |
| | AGTARAVSEFGTIMSISAYSG | |
| | ATFEEISEGLNGGPRWFQIYM | |
| | AKDDQQNRDILDEAKSDGATA | |
| | IILTADSTVSGNRDRDVKNKF | |
| | VYPFGMPIVQRYLRGTAEGMS | |
| | LNNIYGASKQKISPRDIEEIA | |
| | AHSGLPVFVKGIQHPEDADMA | |
| | IKAGASGIWVSNHGARQLYEA | |
| | PGSFDTLPAIAERVNKRVPIV | |
| | FDSGVRRGEHVAKALASGADV | |
| | VALGRPVLFGLALGGWQGAYS | |
| | VLDYFQKDLTRVMQLTGSQNV | |
| | EDLKGLDLFDNPYGYEY | |
| A96C | MNNNDIEYNAPSEIKYIDVVN | 4 |
| | TYDLEEEASKVVPHGGFNYIA | |
| | GASGDEWTKRANDRAWKHKLL | |
| | YPRLAQDVEAPDTSTEILGHK | |
| | IKAPFIMAPIACHGLAHTTKE | |
| | AGTARAVSEFGTIMSISAYSG | |
| | ATFEEISEGLNGGPRWFQIYM | |
| | AKDDQQNRDILDEAKSDGATA | |
| | IILTADSTVSGNRDRDVKNKF | |
| | VYPFGMPIVQRYLRGTAEGMS | |
| | LNNIYGASKQKISPRDIEEIA | |
| | AHSGLPVFVKGIQHPEDADMA | |
| | IKAGASGIWVSNHGARQLYEA | |
| | PGSFDTLPAIAERVNKRVPIV | |
| | FDSGVRRGEHVAKALASGADV | |
| | VALGRPVLFGLALGGWQGAYS | |
| | VLDYFQKDLTRVMQLTGSQNV | |
| | EDLKGLDLFDNPYGYEY | 5 |
| S175C | MNNNDIEYNAPSEIKYIDVVN | |
| | TYDLEEEASKVVPHGGFNYIA | |
| | GASGDEWTKRANDRAWKHKLL | |
| | YPRLAQDVEAPDTSTEIGHKL | |
| | IKAPFIMAPIAAHGLAHTTKE | |
| | AGTARAVSEFGTIMSISAYSG | |
| | ATFEEISEGLNGGPRWFQIYM | |
| | AKDDQQNRDILDEAKSDGATA | |
| | IILTADCTVSGNRDRDVKNKF | |
| | VYPFGMPIVQRYLRGTAEGMS | |
| | LNNIYGASKQKISPRDIEEIA | |
| | AHSGLPVFVKGIQHPEDADMA | |
| | IKAGASGIWVSNHGARQLYEA | |
| | PGSFDTLPAIAERVNKRVPIV | |
| | FDSGVRRGEHVAKALASGADV | |
| | VALGRPVLFLALGGWQGAYSG | |
| | VLDYFQKDLTRVMQLTGSQNV | |
| | EDLKGLDLFDNPYGYEY | |
| S175C- | MNNNDIEYNAPSEIKYIDVVN | 6 |
| carboxy- | TYDLEEEASKVVPHGGFNYIA | |
| methyl- | GASGDEWTKRANDRAWKHKLL | |
| ated | YPRLAQDVEAPDTSTEILGHK | |
| | IKAPFIMAPIAAHGLAHTTKE | |
| | AGTARAVSEFGTIMSISAYSG | |
| | ATFEEISEGLNGGPRWFQIYM | |
| | AKDDQQNRDILDEAKSDGATA | |
| | IILTADC*TVSGNRDRDVKNK | |
| | FVYPFGMPIVQRYLRGTAEGM | |
| | SLNNIYGASKQKISPRDIEEI | |
| | AAHSGLPVFVKGIQHPEDADM | |
| | AIKAGASGIWVSNHGARQLYE | |
| | APGSFDTLPAIAERVNKRVPI | |
| | VFDSGVRRGEHVAKALASGAD | |
| | VVALGRPVLFGLALGGWQGAY | |
| | SVLDYFQKDLTRVMQLTGSQN | |
| | VEDLKGLDLFDNPYGYEY | |

*: Cysteine that is modified with a carboxymethyl group.

### Example 2 Characterization of the Present Lactate Oxidase Variants

### 2.1 Oxidase and Dehydrogenase Activities

The oxidative and dehydrogenase activities of the present lactate oxidase variants were characterized in this example. To this purpose, the wild-type and mutant lactate oxidases of the present application were respectively mixed and reacted with lactic acid (10 mM), and their protein activities were individually calculated in according with procedures described in “Materials and Methods” section. Quantitative results are listed in Table 3.

TABLE 3
Quantitative results of oxidase and dehydrogenase activities
of wild-type (WT) and mutant lactate oxidases

| | Oxidase activity | Dehydrogenase activity |
|---|---|---|
| | (U/mg) | (U/mg) |
| WT | 58.18 | 9.16 |
| A95N | ND | 0.07 |
| A95Q | 0.93 | 0.49 |
| A96C | 17.76 | 1.07 |
| S175C | 18.33 | 1.21 |

*ND: not detected.

The data in Table 3 evidenced that, both oxidase and dehydrogenase activities of the mutant lactate oxidases decreased significantly, as compared to those of the wild-type protein, particularly the oxidase activity and the dehydrogenase activity of A95Q variant were respectively 98.4% and 95% less than those of the wild-type lactate oxidase.

### 2.2 Enzyme Kinetics

In this example, whether the binding affinity of the present lactate oxidase variant was affected by pH was investigated. To this purpose, the present lactate oxidase variants were reacted with various concentrations of lactic acid at pH 5.0 or pH 7.0, and Kₘ and K_{cat} values of enzymes were determined in accordance with procedures described in “Materials and Methods” section. Kₘ and K_{cat} values are listed in Table 4.

TABLE 4
Kₘ and K_{cat} values of the present lactate oxidase variants and wild-type (WT) enzyme

| | | pH 5 | | | pH 7 | | |
|---|---|---|---|---|---|---|---|
| | | Kₘ | K_{cat} | K_{cat}/Kₘ | Kₘ | K_{cat} | K_{cat}/Kₘ |
| Chemical reaction | | (mM) | (S⁻¹) | (mM⁻¹S⁻¹) | (mM) | (S⁻¹) | (mM⁻¹S⁻¹) |
| Oxidation | WT | 2.47 | 10.47 | 4.25 | 0.45 | 10.56 | 23.5 |
| | A95N | ND | ND | ND | ND | ND | ND |
| | A95Q | 34.07 | 0.32 | 0.01 | 12.24 | 0.48 | 0.04 |
| | A96C | 22.65 | 4.63 | 0.2 | 10.44 | 7.84 | 0.75 |
| | S175C | 17.22 | 9.16 | 0.53 | 2.38 | 5.56 | 2.33 |
| Dehydrogenation | WT | 1.51 | 2.45 | 1.63 | 0.29 | 3.23 | 11.1 |
| | A95N | 551.5 | 0.91 | 0.0017 | 1464 | 4.72 | 0.0032 |
| | A95Q | 6.63 | 0.045 | 0.0068 | 10.31 | 0.17 | 0.02 |
| | A96C | 17.94 | 0.67 | 0.038 | 8.81 | 1.48 | 0.17 |
| | S175C | 5.94 | 1.00 | 0.17 | 3.07 | 4.43 | 1.44 |

*ND: not detected.

It was found that, regardless the changes in pH value (i.e., pH 5.0 or pH 7.0), Kₘ values of the present lactate oxidase variants were significantly higher than those of wild-type enzyme, indicating that each of the present lactate oxidase variants had lower binding affinity towards lactic acid.

### Example 3 Lactate Detection Via Use of the Present Lactate Oxidase Variants

In this example, the sensitivity and versatility of the present lactate oxidase variants for lactate detection in various samples were evaluated. To this purpose, two types of lactate sensors (LS-I and LS-II) were constructed based on different types of redox mediators fixed on the screen-printed carbon electrode (SPCE) in accordance with procedures described in “Materials and Methods” section. Specifically, LS-I was produced by depositing the mixture of enzyme solutions (i.e., the present lactate oxidase variants A95Q, A96C, S175C, and carboxymethylated S175C) and the high polymer mediator PVImQOs on the surface of SPCE; and LS-II was produced by immobilizing the enzyme and potassium ferricyanide (K₃[Fe(CN)₆]) onto the surface of SPCE. LS-I and LS-II were respectively used to detect lactate in designated samples.

### 3.1 Binding Affinity

Whether the binding affinities of the present lactate oxidase variants affected by pH was investigated in this experiment. To this purpose, the reaction between lactic acid (at various concentrations) and the present lactate oxidase variants at pH 5.0 or pH 7.0 was determined by the lactate sensor LS-I. Kₘ and Vₘₐₓ values of the enzymes (i.e., the present lactate oxidase variants) were determined based on procedures described in “Materials and Methods” section, and results are summarized in Table 5.

TABLE 5
Kₘ and Vₘₐₓ values of the wild-type (WT)
and the present mutant lactate oxidases

| | |
|---|---|
| pH 5.0 | pH 7.0 |

| | Kₘ (mM) | Vₘₐₓ (μA) | Kₘ (mM) | Vₘₐₓ (μA) |
|---|---|---|---|---|
| WT | 1.84 | 25.29 | 2.33 | 41.14 |
| A95N | 6.87 | 0.087 | 255.5 | 3.45 |
| A95Q | 24.12 | 0.019 | 9.47 | 0.22 |
| A96C | 3.28 | 0.095 | 2.08 | 0.99 |
| S175C | 1.67 | 6.16 | 1.31 | 11.78 |
| S175C** | 3.27 | 0.27 | 17.16 | 1.39 |

**Cysteine at position 175 is modified with a carboxymethyl group

It was found that, as summarized in Table 5, the present lactate oxidase variants had Kₘ values larger than that of the wildtype enzyme. The data in Table 5 is in line with previous assessments in Example 2, both indicating that the binding affinities of the present lactate oxidase variants to lactate are lower than that of the wild-type lactate oxidase, particularly in lower pH environment.

### 3.2 Lactate Detection Via Use of the Present Lactate Oxidase Variants

### 3.2.1 A95Q, A96C, or S175C

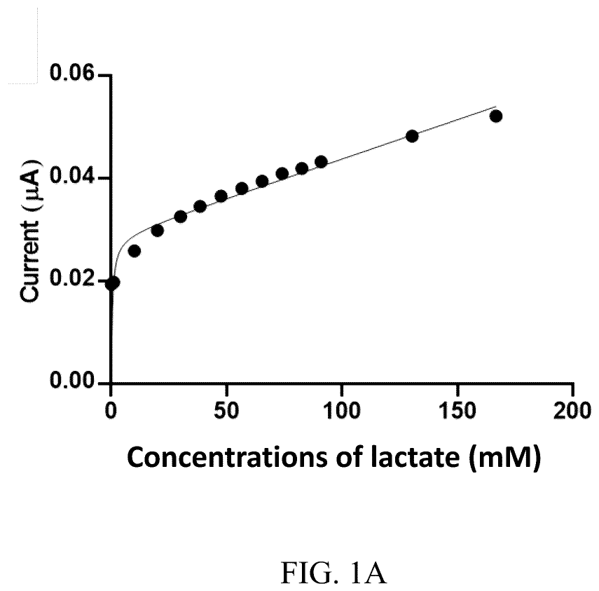

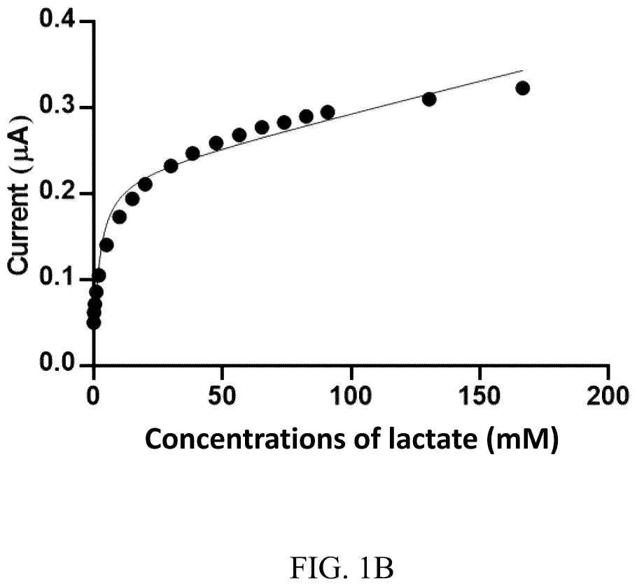

In this experiment, the detection efficiency and limitation of the present mutant lactate oxidase A95Q, A96C and S175C were investigated. To this purpose, the present mutant lactate oxidase A95Q, A96C or S175C within the mediator PVImQOs was independently immobilized onto the surface of SPCE, thereby producing a type-I lactate sensor (hereinafter, A95Q-LS-I, A96C-LS-I, and S175C-LS-I). Then, various concentrations of lactic acid (0-300 mM) in phosphate buffer (pH 5 and pH 7) or synthetic sweat (pH 5) were respectively applied onto each lactate sensors, and the thus-produced currents were measured and recorded in accordance with the procedures as described in the “Materials and Methods” section. Results were provided in FIGS. 1 to 3.

It was found that, regardless of the pH of the buffer solution, the present A95Q enzyme could successfully detect different concentrations of lactic acid, and the minimum and maximum concentrations were about 0.2 mM and 166 mM, respectively. Data depicted in FIGS. 1A and 1B collectively indicated that the present lactate oxidase A95Q variant was able to detect a wide range of lactic acid in liquid sample.

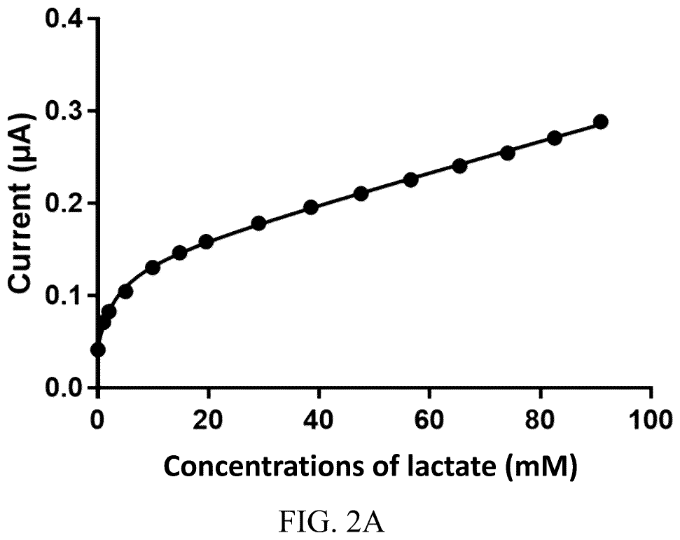

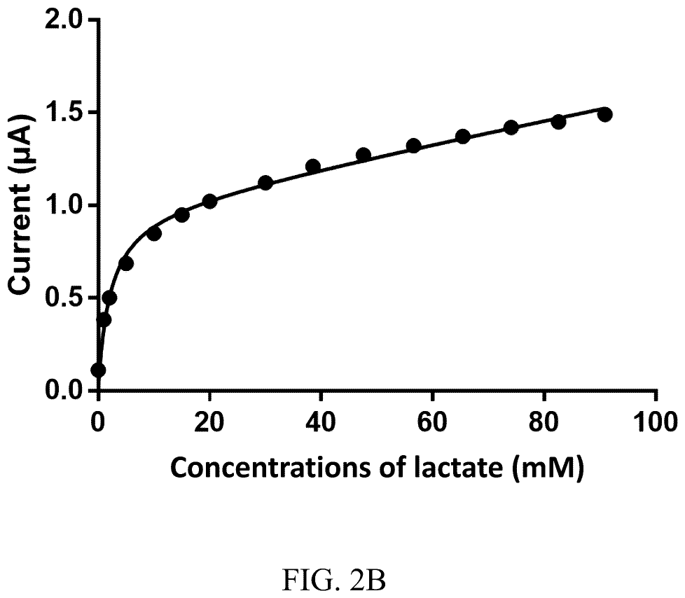

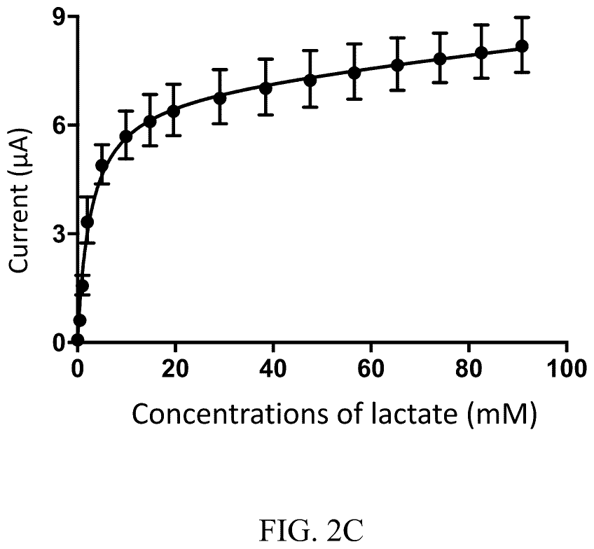

As to A96C, it was found that A96C could detect lactic acid ranging from about 0.5 mM to 90 mM, either in pH 5.0 (FIG. 2A) or pH 7.0 (FIG. 2B) buffer solutions. Consistent result was also observed in the detection of lactate in synthetic sweat (pH 5.0, FIG. 2C). In the synthetic sweat, the detectable lactate concentration ranged from 0.5 mM to 90 mM, indicating that the present lactate oxidase A96C is capable of detecting lactate in the simulated bio-fluid environment.

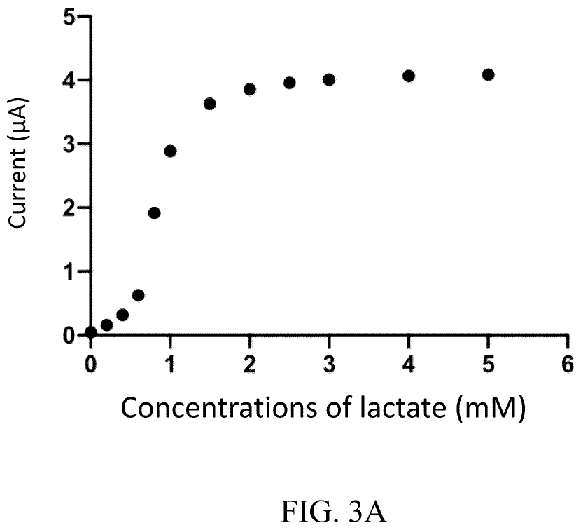

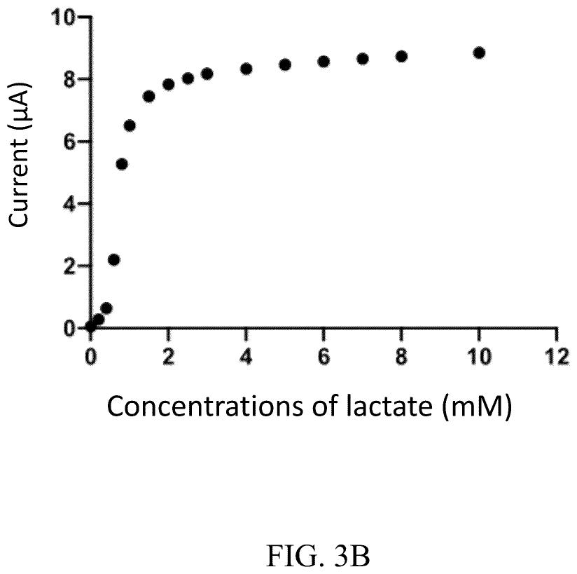

For S175C, it was found that trace concentrations of lactic acid (i.e., 0.2 mM to 10 mM) were detected by the present lactate oxidase variant S175C, irrespective of the pH value of the buffer solution (FIGS. 3A and 3B). The results indicated that the present lactate oxidase variant S175C can detect and quantify lactate in small scale (e.g., lower than 10 mM in the present disclosure) in aqueous samples without being interfered by different pH levels.

### 3.2.2 Carboxymethylated S175C

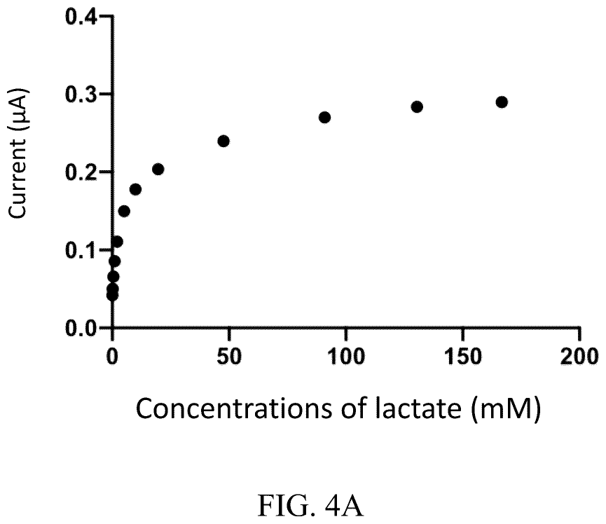

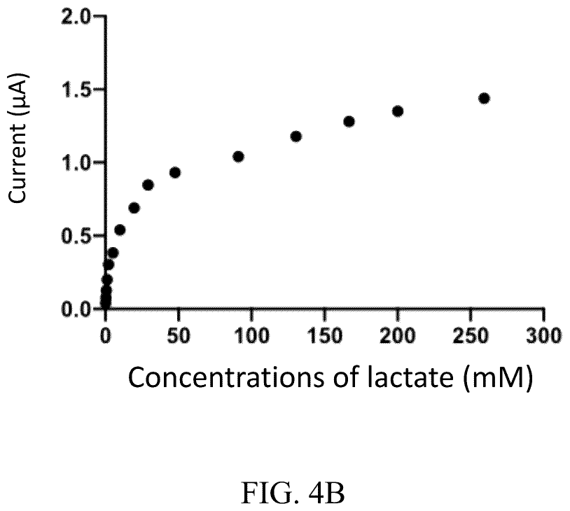

In this experiment, the S175C variant was reacted with 0.1 M of iodoacetic acid (IAA) to produce carboxymethylated S175C, and its effect in lactic acid detection was investigated. Results are depicted in FIGS. 4A and 4B.

As depicted, the carboxymethylation greatly improved the detection range of lactic acid of the S175C variant, as compared to that of the control variant. Specifically, the maximum concentrations of lactic acid in the acid buffer solution (pH 5.0) and the neutral buffer solutions (pH 7.0) detectable by the carboxymethylated variant respectively went up to 166.7 mM, and 259.3 mM.

### 3.3 Versatility of the Present Lactate Oxidase Variant in Lactate Detection

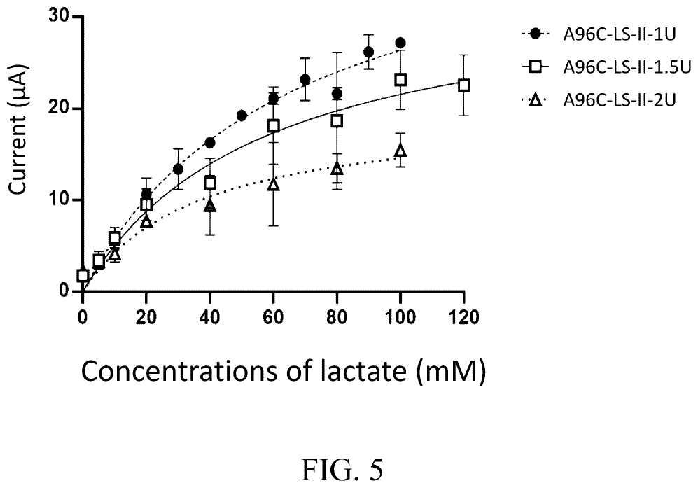

In this example, the versatility of the present lactate oxidase variants in lactate detection were investigated via varying the amount of enzyme being immobilized on the detection electrode, the pH value and/or salinity in the buffer solution. Results are depicted in FIGS. 5 to 7.

It was found that, the maximum amount of lactate that could be detected by sensors immobilized with 1, 1.5 or 2 units (U) of A96C had exceeded 100 mM, particularly, the lactate sensor immobilized with 1.5U of A96C (i.e., A96C-LS-II-1.5U) exhibited the maximal detection power, in which the detectable lactate concentration in synthetic sweat was 120 mM (FIG. 5). Further, the sensor immobilized with 1 unit of A96C (i.e., A96C-LS-II-1U) exhibited highest resolution and less variability (FIG. 5), which indicates that only a small amount of mutant enzyme was required to achieve desired detection efficacy.

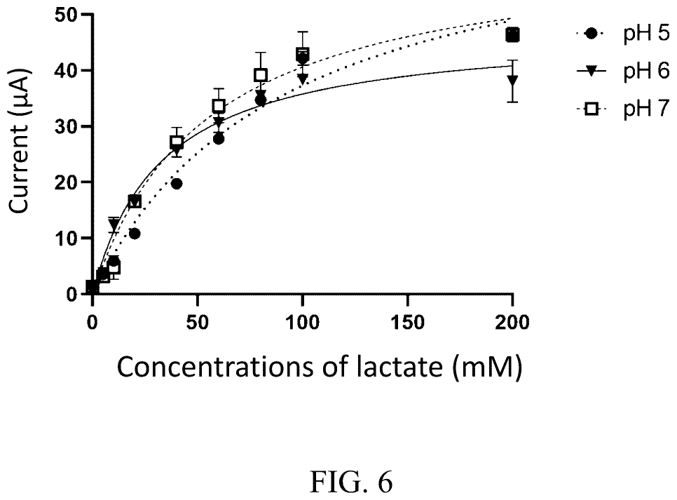

As to the effect of pH in lactate detection, it was found that, regardless of the pH level, the present mutant lactate oxidase A96C was capable of detecting lactate ranging from 0 to 200 mM in the sweat samples (FIG. 6).

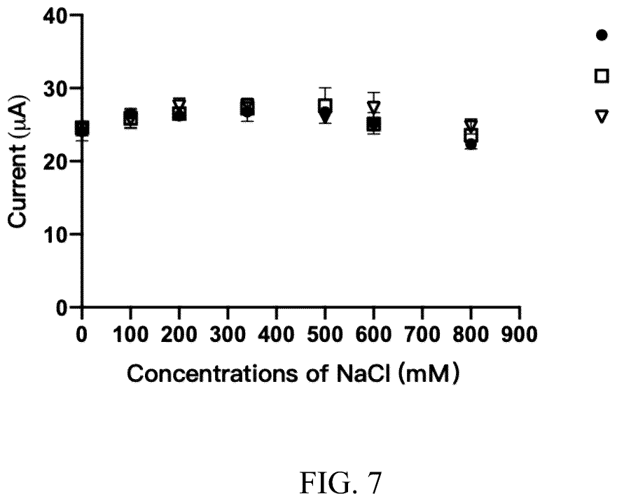

As to the effect of salinity, only small differences in currents generated upon the reaction of lactate and the present lactate oxidase A96C variant in various pH levels and/or salinities were found (FIG. 7). The data depicted in FIG. 7 confirmed that the detection efficacy of the present lactate oxidase A96C was unaffected or interfered by salinity of synthetic sweat.

Taken together, the data of Examples 1 to 3 collectively indicate that the present lactate oxidase variants do possess improved lactate detection efficacy without being interfered by pH level and/or salinity of liquid samples, so as to achieve a wider versatility in lactate detection.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A lactate oxidase variant derived from a wild-type lactate oxidase of SEQ ID NO:1, wherein the lactate oxidase variant comprises an amino acid substitution at positions 95, 96, or 175 of the SEQ ID NO:1, or a combination thereof, in which
alanine (A) at position 95 of the SEQ ID NO:1 is substituted by asparagine (N) or glutamine (Q);
alanine (A) at position 96 of the SEQ ID NO: 1 is substituted by cysteine (C); and/or seine(S) at position 175 of the SEQ ID NO:1 is substituted by cysteine (C).

2. The lactate oxidase variant of claim 1, wherein the alanine (A) at position 95 of the SEQ ID NO: 1 is substituted by asparagine (N).

3. The lactate oxidase variant of claim 1, wherein the alanine (A) at position 95 of the SEQ ID NO: 1 is substituted by glutamine (Q).

4. The lactate oxidase variant of claim 1, wherein the alanine (A) at position 96 of the SEQ ID NO: 1 is substituted by cysteine (C).

5. The lactate oxidase variant of claim 1, wherein the seine(S) at position 175 of the SEQ ID NO: 1 is substituted by cysteine (C).

6. The lactate oxidase variant of claim 5, wherein the cysteine (C) at position 175 of the SEQ ID NO: 1 is carboxymethylated.

7. The lactate oxidase variant of claim 1, wherein the lactate oxidase variant has the amino acid sequence of SEQ ID NO: 2, 3, 4, 5, or 6.

8. The lactate oxidase variant of claim 1, wherein the lactate oxidase variant has a binding affinity to lactate lower than that of the wild-type lactate oxidase.

9. A method for detecting and quantifying lactate in a liquid sample, comprising,
(a) contacting the liquid sample with a lactate oxidase variant of claim 1;
(b) measuring a current generated by the reaction between the lactate oxidase variant of claim 1 and the lactate in the liquid sample; and
(c) determining the concentration of lactate in the liquid sample via interpolating or extrapolating the current measured in step (b) with that of a control sample having a known concentration of lactate.

10. The method of claim 9, wherein the liquid sample has a pH value ranging from 4 to 9.

11. The method of claim 9, wherein the liquid sample has a salinity between 0 to 1000 mM.

12. The method of claim 9, wherein the liquid sample is sweat.

13. The method of claim 9, wherein the method is capable of detecting lactate ranging from 0 to 300 mM in the liquid sample.


SEQUENCE LISTING (14 sequences)

SEQ ID NO: 1 - protein, 374 aa, Aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIAAHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 2 - protein, 374 aa, synthetic construct
  REGION 1..374: note = Singel substitution at postiion 95 of the SEQ ID NO:1
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPINAHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 3 - protein, 374 aa, synthetic construct
  REGION 1..374: note = Single substitution at position 95 of the SEQ ID NO:1
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIQAHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 4 - protein, 374 aa, synthetic construct
  REGION 1..374: note = Single substitution at position 96 of the SEQ ID NO:1
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIACHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 5 - protein, 374 aa, synthetic construct
  REGION 1..374: note = Single substitution at position 175 of the SEQ ID NO:1
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIAAHGLAHTTKEAGTARAVSEFGTIMSISAYSGATFEEISEGLNGGPRWFQIYMAKDDQQNRDILDEAKSDGATAIILTADCTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 6 - protein, 374 aa, synthetic construct; identical to SEQ ID NO: 5
  REGION 1..374: note = The cysteine (C) at position 175 of the SEQ ID NO:5 iscarboxymethylated
  UNSURE 175: note = Carboxylmethylation

SEQ ID NO: 7 - DNA, 44 bp, synthetic construct
  misc_feature 1..44: note = Forward primer for lactate oxidase mutant A95N
  source 1..44: mol_type = other DNA
ccgtttattatggcaccgattaacgcacatggtctggcacatac

SEQ ID NO: 8 - DNA, 44 bp, synthetic construct
  misc_feature 1..44: note = Reverse primer for lactate oxidase mutant A95N
  source 1..44: mol_type = other DNA
gtatgtgccagaccatgtgcgttaatcggtgccataataaacgg

SEQ ID NO: 9 - DNA, 41 bp, synthetic construct
  misc_feature 1..41: note = Forward primer for lactate oxidase mutant A95Q
  source 1..41: mol_type = other DNA
cgtttattatggcaccgattcaagcacatggtctggcacat

SEQ ID NO: 10 - DNA, 41 bp, synthetic construct
  misc_feature 1..41: note = Reverse primer for lactate oxidase mutant A95Q
  source 1..41: mol_type = other DNA
atgtgccagaccatgtgcttgaatcggtgccataataaacg

SEQ ID NO: 11 - DNA, 40 bp, synthetic construct
  misc_feature 1..40: note = Forward primer for lactate oxidase mutant A96C
  source 1..40: mol_type = other DNA
ttatggcaccgattgcatgccatggtctggcacataccac

SEQ ID NO: 12 - DNA, 40 bp, synthetic construct
  misc_feature 1..40: note = Reverse primer for lactate oxidase mutant A96C
  source 1..40: mol_type = other DNA
gtggtatgtgccagaccatggcatgcaatcggtgccataa

SEQ ID NO: 13 - DNA, 35 bp, synthetic construct
  misc_feature 1..35: note = Forward primer for lactate oxidase mutant S175C
  source 1..35: mol_type = other DNA
tattctgaccgcagattgcaccgttagcggtaatc

SEQ ID NO: 14 - DNA, 35 bp, synthetic construct
  misc_feature 1..35: note = Reverse primer for lactate oxidase mutant S175C
  source 1..35: mol_type = other DNA
gattaccgctaacggtgcaatctgcggtcagaata